(19) United States
(12) Patent Application Publication
(10) Pub. No.: US 2023/0302304 A1
(43) Pub. Date: Sep. 28, 2023

(54) PORTABLE VESSEL, REGULATOR AND APPARATUS FOR STORING FLUIDS

(71) Applicant: Jarvis B. Jenkins, Jacksonville Beach, FL (US)

(72) Inventor: Jarvis B. Jenkins, Jacksonville Beach, FL (US)

(21) Appl. No.: 18/022,231

(22) PCT Filed: Aug. 20, 2021
(86) PCT No.: PCT/US2021/046797

§ 371 Date: Feb. 20, 2023

Related U.S. Application Data
(60) Provisional application No. 63/068,828, filed on Aug. 21, 2020.

(51) Int. Cl. A62B 7/02; F17C 1/14; A61M 16/20; B63C 11/22; A62B 9/02

(52) U.S. Cl. CPC A62B 7/02; F17C 1/14; A61M 16/20; B63C 11/2209; A62B 9/02; F17C 2201/058; F17C 2270/025; F17C 2203/0643; F17C 2223/036; F17C 2201/0109; F17C 2205/0338; F17C 2221/031; F17C 2221/011; F17C 2221/012; F17C 2221/014; F17C 2221/017; B63C 11/2227; B63C 2011/2218

(57) ABSTRACT

Portable vessels, regulators, apparatus, and methods for storing fluids under pressure are disclosed.

This application is being filed as a PCT International Patent Application in the name of Jarvis B. Jenkins, a U.S. citizen, claiming priority to U.S. Provisional Patent Application Ser. No. 63/068,828 filed on 21 Aug. 2020 and entitled “PORTABLE VESSEL, REGULATOR AND APPARATUS FOR STORING FLUIDS.”

### FIELD OF THE INVENTION

The present invention is directed to at least one portable vessel and a pressure regulator, and an apparatus that includes the at least one portable vessel and the pressure regulator, which are capable of storing fluids under pressure. The present invention is also directed to a method of compressing a fluid in at least one portable vessel to a high level; and a further method of releasing compressed fluid at a high level from an apparatus including at least one of a portable vessel and a pressure regulator.

### BACKGROUND OF THE INVENTION

There is a need in the art for portable vessels, that store fluids under pressure, and pressure regulators, that are compact, and still manage to contain and dispense a substantial capacity of fluid. There is also a need in the art for a portable apparatus that is compact and capable of effectively providing a substantial capacity of fluid.

### SUMMARY OF THE INVENTION

The present invention relates to the discovery of at least one of a portable vessel and a pressure regulator, and an apparatus including the same, which are capable of storing fluid under pressure. The disclosed at least one of a portable vessel and pressure regulator, and apparatus comprising the same, provide a number of advantages over known potable vessels and pressure regulators, and apparatus comprising the same. For example, the disclosed at least one of a portable vessel and pressure regulator, and apparatus comprising the same, are compact and operate at higher pressures, which increases the capacity of the vessel and apparatus. In addition, the disclosed at least one of a portable vessel and pressure regulator, and apparatus comprising the same, allow(s) the use of smaller portable vessels, which are significantly more convenient to handle.

The present invention also relates to the discovery that at least one of a portable vessel and pressure regulator, and apparatus including the same, for storing fluids under pressure, are comprised of a certain stainless steel. In one exemplary embodiment, the present invention is directed to at least one of a portable vessel and pressure regulator, and apparatus including the same for storing fluids under pressure, which comprise(s) stainless steel, wherein the stainless steel includes at least chromium in an amount of about 17.0 to about 40.0 weight percent (wt %); molybdenum in an amount of about 0.01 to about 10.00 wt %; and nickel in an amount of about 1.0 to about 12.0 wt %; or in a further exemplary embodiment, the stainless steel of the present invention includes at least chromium in an amount of about 20 to about 40 wt %; molybdenum in an amount of about 0.05 to about 10.00 wt %; and nickel in an amount of about 1.5 to about 12.0 wt %. In another exemplary embodiment, the stainless steel of the present invention includes at least carbon in an amount of 0.0100-0.0800 wt %; chromium in an amount of 18.5-38.5 wt %; copper in an amount of 0.0500-5.30 wt %; iron in an amount of 55.2-72.7 wt %; manganese in an amount of 0.400-5.00 wt %; molybdenum in an amount of 0.0500-7.00 wt %; nickel in an amount of 1.50-11.0 wt %; nitrogen in an amount of 0.0500-0.720 wt %; phosphorous in an amount of 0.0150-0.0500 wt %; silicon in amount of 0.200-2.60 wt %; and sulfur in an amount of 0.00010-0.0400 wt %, with the balance being naturally occurring impurities.

In another exemplary embodiment, the present invention is directed to at least one of a portable vessel and pressure regulator, and apparatus including the same, for storing fluids under pressure, which comprise(s) stainless steel, wherein the stainless steel (1) comprises ferritic and austenitic phases, and (2) comprises at least the following: chromium in an amount of about 17.0 to about 40.0 weight percent (wt %); molybdenum in an amount of about 0.01 to about 10.00 wt %; and nickel in an amount of about 1.0 to about 12.0 wt %, based on a total weight of the stainless steel.

In another exemplary embodiment, the present invention is directed to at least one of a portable vessel and pressure regulator, and apparatus including the same, for storing fluids under pressure, which comprise(s) stainless steel, wherein the stainless steel possesses a tensile strength of at least about 90 ksi.

In a further exemplary embodiment, the present invention is directed to at least one of a portable vessel and pressure regulator, and apparatus including the same, for storing fluids under pressure, which comprise(s) stainless steel, wherein the stainless steel possesses a critical pitting corrosion temperature according to ASTM G48C of at least about 80° C.

In another exemplary embodiment, the present invention is directed to at least one of a portable vessel and pressure regulator, and apparatus including the same for storing fluids under pressure, which comprise(s) stainless steel, wherein the stainless steel possesses a pitting resistance equivalent number of at least about 35.

In one exemplary embodiment, the present invention is directed to at least one of a portable vessel and a pressure regulator, and apparatus including the same, for storing fluids under pressure, which comprise(s) stainless steel, wherein the stainless steel possesses an impact resistance of at least about 100 Joules (J) at room temperature.

In another exemplary embodiment, the present invention is directed to at least one of a portable vessel and pressure regulator, and apparatus including the same, for storing fluids under pressure, which comprise(s) stainless steel, wherein the portable vessel and pressure regulator are capable of a service pressure of at least about 5000 pounds per square inch (psi) and a burst pressure of at least about 10,000 psi.

In a further exemplary embodiment, the present invention is directed to at least one portable vessel and pressure regulator, and apparatus including the same, for storing fluids under pressure, which comprise(s) stainless steel, wherein the vessel is cylindrical in shape with closed ends and wherein the vessel is capable of holding at least about 80 ft³ of a gas, has a length of about 25 inches or less and an outer diameter of about 8 inches or less.

In another exemplary embodiment, the present invention is directed to an apparatus including at least one portable vessel that is cylindrical in shape with closed ends, with at least one end having an aperture for receiving a valve boss; and at least one pressure regulator, which reduces the pressure from the portable vessel to a predetermined level; wherein at least one of the portable vessel and pressure regulator comprise(s) a certain stainless steel. In one exemplary embodiment, the stainless steel is comprised of at least chromium in an amount of about 17.0 to about 40.0 wt %; molybdenumin an amount of about 0.01 to about 10.00 wt %; and nickel in an amount of about 1.0 to about 12.0 wt %; or in a further exemplary embodiment, the stainless steel of the present invention includes at least chromium in an amount of about 20.0 to about 40.0 wt %; molybdenum in an amount of about 0.05 to about 10.00 wt %; and nickel in an amount of about 1.5 to about 12.0 wt %. In another exemplary embodiment, the stainless steel of the present invention includes at least carbon in an amount of 0.0100-0.0800 wt %; chromium in an amount of 18.5-38.5 wt %; copper in an amount of 0.0500-5.30 wt %; iron in an amount of 55.2-72.7 wt %; manganese in an amount of 0.400-5.00 wt %; molybdenum in an amount of 0.0500-7.00 wt %; nickel in an amount of 1.50-11.0 wt %; nitrogen in an amount of 0.0500-0.720 wt %; phosphorous in an amount of 0.0150-0.0500 wt %; silicon in an amount of 0.200-2.60 wt %; and sulfur in an amount of 0.00010-0.0400 wt %, with the balance being naturally occurring impurities. In another exemplary embodiment, the stainless steel possesses at least one of: a tensile strength of at least about 90 ksi, a critical pitting corrosion temperature according to ASTM G48C of at least about 80°, a pitting resistance equivalent number of at least about 35, and an impact resistance of at least about 100 J at room temperature. In a further exemplary embodiment, the vessel is capable of a service pressure of at least about 4000 psi and a burst pressure of at least about 9,000 psi. In an even further exemplary embodiment, the portable vessel is capable of holding at least about 80 ft³ of a gas, has a length of about 25 inches or less and an outer diameter of about 8 inches or less.

In another exemplary embodiment, the present invention is directed to an apparatus for storing fluids under pressure, the apparatus including at least one first portable vessel that is cylindrical in shape with closed ends, with at least one end having an aperture for receiving a valve boss; and at least one second portable vessel that is cylindrical in shape with closed ends, with at least one end having an aperture for receiving a valve boss, the at least one second portable vessel being different in shape or size from the at least one first portable vessel; wherein at least one of the first portable vessel and second portable vessel comprise(s) certain stainless steel. In one exemplary embodiment, the stainless steel is comprised of at least chromium in an amount of about 17.0 to about 40.0 wt %; molybdenum in an amount of about 0.01 to about 10.00 wt %; and nickel in an amount of about 1.0 to about 12.0 wt %; or in a further exemplary embodiment, the stainless steel of the present invention includes at least chromium in an amount of about 20.0 to about 40.0 wt %; molybdenum in an amount of about 0.05 to about 10.00 wt %; and nickel in an amount of about 1.5 to about 12.0 wt %. In another exemplary embodiment, the stainless steel of the present invention includes at least carbon in an amount of 0.0100-0.0800 wt %; chromium in an amount of 18.5-38.5 wt %; copper in an amount of 0.0500-5.30 wt %; iron in an amount of 55.2-72.7 wt %; manganese in an amount of 0.400-5.00 wt %; molybdenum in an amount of 0.0500-7.00 wt %; nickel in an amount of 1.50-11.0 wt %; nitrogen in an amount of 0.0500-0.720 wt %; phosphorous in an amount of 0.0150-0.0500 wt %; silicon in amount of 0.200-2.60 wt %; and sulfur in an amount of 0.00010-0.0400 wt %, with the balance being naturally occurring impurities. In another exemplary embodiment, the stainless steel possesses at least one of: a tensile strength of at least about 90 ksi, a critical pitting corrosion temperature according to ASTMG48C of at least about 80°, a pitting resistance equivalent number of at least about 35, and an impact resistance of at least about 100 J at room temperature. In a further exemplary embodiment, the vessel and at least one additional vessel are capable of a service pressure of at least about 5000 psi and a burst pressure of at least about 10,000 psi. In an even further exemplary embodiment, the portable vessel is capable of holding at least about 80 ft³ of a gas, has a length of about 25 inches or less and an outer diameter of about 8 inches or less and the at least one additional vessel is capable of holding at least about 20 ft³ of a gas, has a length of about 10 inches or less and an outer diameter of 5 about inches or less.

In another exemplary embodiment, the present invention is directed to a pressure regulator for reducing pressure from a portable vessel for storing fluids under pressure, the pressure regulator having a valve with a valve body comprised of stainless steel, to be in fluid communication with said vessel, which reduces the pressure from the vessel to a predetermined level. In one exemplary embodiment, the stainless steel is comprised of at least chromium in an amount of about 17.0 to about 40.0 wt %; molybdenumin an amount of about 0.01 to about 10.00 wt %; and nickel in an amount of about 1.0 to about 12.0 wt %; or in a further exemplary embodiment, the stainless steel of the present invention includes at least chromium in an amount of about 20.0 to about 40.0 wt %; molybdenum in an amount of about 0.05 to about 10.00 wt %; and nickel in an amount of about 1.5 to about 12.0 wt %. In another exemplary embodiment, the stainless steel of the present invention includes at least carbon in an amount of 0.0100-0.0800 wt %; chromium in an amount of 18.5-38.5 wt %; copper in an amount of 0.0500-5.30 wt %; iron in an amount of 55.2-72.7 wt %; manganese in an amount of 0.400-5.00 wt %; molybdenum in an amount of 0.0500-7.00 wt %; nickel in an amount of 1.50-11.0 wt %; nitrogen in an amount of 0.0500-0.720 wt %; phosphorous in an amount of 0.0150-0.0500 wt %; silicon in amount of 0.200-2.60 wt %; and sulfur in an amount of 0.00010-0.0400 wt %, with the balance being naturally occurring impurities. In another exemplary embodiment, the stainless steel possesses at least one of: a tensile strength of at least about 90 ksi, a critical pitting corrosion temperature according to ASTM G48C of at least about 80°, a pitting resistance equivalent number of at least about 35, and an impact resistance of at least about 100 J at room temperature. In a further exemplary embodiment, the vessel and regulator are capable of a service pressure of at least about 4000, 4500 or 5000 psi, and a burst pressure of at least about 9,000, 9,500 or 10,000 psi. In an even further exemplary embodiment, the portable vessel of the present invention is capable of holding at least about 80 ft³ of a gas, has a length of about 25 inches or less and an outer diameter of about 8 inches or less. In another exemplary embodiment, the pressure regulator comprised of the subject stainless steel is capable of reducing pressure from the portable vessel at a pressure of least about 5000 psi to a pressure of less than about 1800 psi.

The present invention is also directed to a method of storing fluid under high pressure in portable equipment; and a second method of releasing fluids from high pressure portable equipment to a predetermined regulated pressure. In one exemplary embodiment, the present invention relates to a method of storing fluid in a portable vessel, including (a) filling the fluid into the portable vessel composed of stainless steel being cylindrical in shape with closed ends, with at least one end having an aperture for receiving a valve boss; and (b) closing the valve after the fluid in the vessel reaches a pressure of at least about 4,000 psi, 4500, 5,000, 5,500 or 6,000 psi. A further exemplary embodiment of the present invention relates to releasing fluid under a pressure of at least about 4,000 psi, 4500, 5,000, 5,500 or 6,000 psi from a portable apparatus comprising at least one of a portable vessel and a pressure regulator made from stainless steel, wherein the pressure regulator reduces the pressure of at least about 4,000 psi, 4500, 5,000, 5,500 or 6,000 psi from the portable vessel to a lower predetermined pressure of about 180 psi or less.

These and other features and advantages of the present invention will become apparent after a review of the following detailed description of the disclosed exemplary embodiments and the appended claims.

### BRIEF DESCRIPTION OF THE FIGURES

FIG. 1 depicts an exemplary portable vessel of the present invention comprising stainless steel;

FIG. 2 depicts an exemplary pressure regulator of the present invention comprising stainless steel; and

FIG. 3 depicts an exemplary portable apparatus of the present invention comprising one or more portable vessel and one or more pressure regulator.

### DETAILED DESCRIPTION OF THE INVENTION

To promote an understanding of the principles of the present invention, descriptions of specific exemplary embodiments of the invention follow, and specific language is used to describe the specific exemplary embodiments. It will nevertheless be understood that no limitation of the scope of the invention is intended by the use of specific language. Alterations, further modifications, and such further applications of the principles of the present invention discussed are contemplated, as would normally occur to one ordinarily skilled in the art to which the invention pertains. Also, it intended that the phraseology and terminology used herein is for the purpose of description and should not be regarded as limiting.

It must be noted that as used herein and in the appended claims, the singular forms “a”, “and”, and “the” include plural referents unless the context clearly dictates otherwise. Thus, for example, reference to “a vessel” includes a plurality of such vessels and reference to “vessel” includes reference to at least one vessels and equivalents thereof known to those skilled in the art, and so forth.

As used herein, the use of the terms “including”, “comprising”, or “having” and variations thereof is meant to encompass the embodiments set forth herein and equivalents thereof, as well as additional components.

“About” modifying, for example, the quantity of an ingredient in a composition, concentrations, volumes, process temperatures, process times, properties, pressures, and like values, and ranges thereof, employed in describing the exemplary embodiments of the disclosure, refers to variation in the numerical quantity that may occur, for example, through typical measuring and handling procedures; through inadvertent error in these procedures; through differences in the ingredients used to carryout the methods; and like proximate considerations. The term “about” also encompasses amounts that differ due to aging of a formulation with a particular initial concentration or mixture, and amounts that differ due to mixing or processing a formulation with a particular initial concentration or mixture. Whether modified by the term “about” the claims appended hereto include equivalents to these quantities.

As used herein, the term “substantially” means within a reasonable amount, but includes amounts which vary from about 0% to about 50% of the absolute value, from about 0% to about 40%, from about 0% to about 30%, from about 0% to about 20% or from about 0% to about 10%.

As used herein, the term “fluid” means at least one of a gas, liquid, and supercritical fluid, or a combination thereof. In exemplary embodiments according to the present invention, the fluid is a gas, including, but not limited to, diving gases, SCBA breathing gases, hypobaric breathing gases and medical breathing gases; such as air, oxygen, hydrogen, nitrogen, helium, neon, nitrox, trimix, heliox, heliair, hydreliox, hydrox, neox, and mixtures thereof.

As used herein, the term “stainless steel” means a family of iron-based alloys that contain a minimum of approximately 11.0 wt % chromium, a composition that prevents the iron from rusting, as well as providing heat-resistant properties. Different types of stainless steel include the elements carbon (from 0.03% to greater than 1.00%), nitrogen, aluminum, silicon, sulfur, titanium, nickel, copper, selenium, niobium, and molybdenum. Specific types of stainless steel are often designated by a three-digit number, e.g., 304 stainless. Stainless steel's resistance to ferric oxide formation results from the presence of chromium in the alloy, which forms a passive film that protects the underlying material from corrosion attack, and can self-heal in the presence of oxygen.

As used herein, the term “portable” means a device or apparatus that is light and small enough so that it may be carried or transported by hand.

As used herein, the term “vessel” means a container capable of storing fluids, including, but not limited to, a cylinder, tank, or bottle.

As used herein, the term “yield strength” means the stress corresponding to the yield point at which a material begins to deform plastically. The term “proof stress” is the stress point at which 0.2% plastic deformation occurs. The term “tensile strength” means the stress corresponding to maximum stress applied to a material at its fracturing point.

As used herein, the term “critical pitting corrosion” means the localized corrosion of a metal surface confined to a point or small area, that takes the form of cavities or pits. The term “critical pitting temperature” is the temperature at which point the pitting test is interrupted according to the test ASTM G48 A.

As used herein, the term “pitting resistance equivalent number” means the parameter for comparing the resistance of stainless steel to pitting in chloride environments and is designated as the PRE number.

As used herein, the term “impact resistance” means the ability of a material to withstand intense force, or its ability to absorb energy during deformation, also known as toughness. As used herein, the impact resistance is measured by the Charpy test under ASTM E23.

As used herein, the term “service pressure” means the authorized pressure marking on the package to which the cylinder may be charged, as defined in 49 C.F.R. § titled “Shippers-General Requirements for Shipments and Packagings,” the entire subject matter of which is incorporated herein by reference.

As used herein, the term “burst pressure” means the pressure at which a vessel ruptures or bursts when conducting a hydrostatic pressure test. A hydrostatic test involves pressurizing the vessel to a pressure (usually 5/3 or 3/2 of the service pressure) and measuring its volume before and after the test. A permanent increase in volume above the tolerated level means the cylinder has reached its “proof pressure” and plastically deformed (i.e., proof stress) and fails the test, and must be permanently removed from service.

As used herein, the terms “mounted”, “connected”, “supported”, and “coupled” and variations thereof are used broadly and include direct and indirect mountings, connections, supports, and couplings. In addition, the terms “connected” and“coupled” are not restricted to physical or mechanical connections or couplings. In the description below, like reference numerals and labels are used to describe the same, similar or corresponding parts in the figures.

The disclosed apparatus is not limited in its application to the details of construction and the arrangement of components set forth in the following description or illustrated in the following drawings. The described apparatus may be used in many applications and in various embodiments. For example, the portable vessel and pressure regulator, or just the portable vessel alone, may be used in any configuration and in numerous fields, including but not limited to, self-contained breathing apparatus (SCBA), self-contained underwater breathing apparatus (SCUBA), medical applications, pneumatic applications, aeronautic applications, mountain climbing applications, sky diving applications, and the like. For example, the portable vessel of the present invention may be used as alternative fuel vessels, industrial gas vessels, CNG vessels, autogas vessels, LNG vessels, SCBA vessels, SCUBA vessels, fire suppression vessels, aviation vessels, acetylene vessels, cryogenic vessels, refrigerant vessels, balloon time helium vessels, and the like.

The present invention relates to the discovery of at least one portable vessel and pressure regulator, and apparatus including the at least one portable vessel and pressure regulator, which are capable of storing fluid under pressure. The disclosed at least one portable vessel, pressure regulator and apparatus provide a number of advantages over known potable vessels and pressure regulators, and apparatus comprising the same. For example, the disclosed at least one portable vessel, pressure regulator and apparatus are compact and operate at higher pressures, which increases the capacity of the vessel and apparatus. In addition, the disclosed at least one portable vessel and pressure regulator, and apparatus comprising the same, allow for the use of smaller portable vessels, which are significantly more convenient to handle. For example, the portable vessels and apparatus of the present invention may be used in small spaces and by operators of small stature. Furthermore, the portable vessels and apparatus may be transported more efficiently, allowing storage in confined spaces. For example, the entire apparatus of the present invention may be stored in a carryon bag used in air travel (e.g., in a container or bag that is 22″×14″×9″ or smaller). In this embodiment of the present invention, the portable vessel of the apparatus is capable of storing at least about 80 ft³ of a gas, has a length of about 25 inches or less and an outer diameter of about 8 inches or less, allowing it to be stored easily in such a small container.

The present invention also relates to the discovery that at least one portable vessel and pressure regulator, and apparatus including the same, for storing fluids under pressure, are comprised of stainless steel. In one exemplary embodiment, the present invention is directed to at least one portable vessel and pressure regulator, and apparatus including the same, for storing fluids under pressure, which comprise stainless steel, wherein the stainless steel includes at least chromium in an amount of about 17.0 to about 40.0 wt %; molybdenum in an amount of about 0.01 to about 10.00 wt %; and nickel in an amount of about 1.0 to about 12.0 wt %; or in a further exemplary embodiment, the stainless steel of the present invention includes at least chromium in an amount of about 20.0 to about 40.0 wt %; molybdenum in an amount of about 0.05 to about 10.00 wt %; and nickel in an amount of about 1.5 to about 12.0 wt %. In another exemplary embodiment, the stainless steel of the present invention includes at least carbon in an amount of 0.0100-0.0800 wt %; chromium in an amount of 18.5-38.5 wt %; copper in an amount of 0.0500-5.30 wt %; iron in an amount of 55.2-72.7 wt %; manganese in an amount of 0.400-5.00 wt %; molybdenum in an amount of 0.0500-7.00 wt %; nickel in an amount of 1.50-11.0 wt %; nitrogen in an amount of 0.0500-0.720 wt %; phosphorous in an amount of 0.0150-0.0500 wt %; silicon in amount of 0.200-2.60 wt %; and sulfur in an amount of 0.00010-0.0400 wt %, with the balance being naturally occurring impurities. In another exemplary embodiment, the stainless steel includes at least the following components: carbon in an amount of 0.0100-0.0800 wt %; chromium in an amount of 20.5-36.5 wt %; copper in an amount of 0.0500-5.30 wt %; iron in an amount of 55.2-72.7 wt %; manganese in an amount of 0.400-5.00 wt %; molybdenum in an amount of 0.0500-7.00 wt %; nickel in an amount of 1.50-11.0 wt %; nitrogen in an amount of 0.0500-0.720 wt %; phosphorous in an amount of 0.0150-0.0500 wt %; silicon in amount of 0.200-2.60 wt %; and sulfur in an amount of 0.00010-0.0400 wt %, with the balance being naturally occurring impurities. The components of the exemplary stainless steel compositions of the present invention are determined using X-ray fluorescence.

In a further exemplary embodiment, the present invention also relates to a stainless steel composition that provides improved chemical properties, such as desirable corrosion resistance. For example, in one embodiment of the present invention, the stainless steel possesses an improved pitting resistance equivalent (PRE) number. The PRE number of stainless steel is a measure of the pitting and crevice corrosion resistance in chloride environments. It is calculated by the formula:

PRE=(wt % Cr)+3.3×(wt % Mo)+16×(wt % N).

In one exemplary embodiment of the present invention, the stainless steel possesses a PRE number of at least about 30, at least about 33, at least about 35, at least about 38, or at least about 40 (or about 30 to about 60, about 33 to about 50, or about 35 to about 50). In another exemplary embodiment of the present invention, the PRE number of the stainless steel is substantially the same in all phases of the steel. For example, if the stainless steel has ferritic and austenitic phases, the PRE number is substantially the same in both of these phases. In an exemplary embodiment, the stainless steel of the present invention is an alloy and is composed of at least one of ferritic and austenitic microstructure, and an even further embodiment, the stainless steel of the present invention is an alloy of ferritic and austenitic microstructure wherein the ferritic microstructure content is about 30.0 to about 70.0 volume percent, or about 40.0 to about 60.0 volume percent of the stainless steel.

In a further exemplary embodiment, the present invention is directed to at least one portable vessel and pressure regulator, and apparatus including the same, for storing fluids under pressure, which comprise stainless steel, wherein the stainless steel is resistant to pitting and crevice corrosion in solutions at higher temperatures. One of the more severe pitting and crevice corrosion tests applied to stainless steel is ASTM G48, which exposes the steel to 6% FeCl₂ solution for a period of time as the temperature is increased. A modified version of ASTM G48 is ASTM G48A. In this test, the stainless steel sample is exposed in the 6% FeCl₂ solution for periods of 24 hours, with the temperature initially set at 0° C. Every 24 hours, the temperature is increased by 5° C. and the test is continued until pits are detected with a substantial weight loss (i.e., more than 5 mg). The test is then discontinued and the critical pitting and crevice corrosion temperature noted. In one exemplary embodiment, the stainless steel of the present invention possesses a critical pitting and crevice corrosion temperature according to ASTM G48A of at least about 60° C., at least about 70° C., at least about 75° C. and at least about 80° C. (or from about 60° C. to about 100° C., from about 60° C. to about 90° C., or from about 70° C. to about 85° C.). This allows the portable vessel and pressure regulator, and the apparatus including the same, be used in a variety of environments, including, but not limited to, high salinity environments.

In another exemplary embodiment, the present invention also relates to a stainless steel composition that provides improved physical properties. For example, in one embodiment of the present invention, the stainless steel possesses a high yield strength and tensile strength, which allows the vessel and pressure regulator to be compact and still operate at high services pressures (e.g., above about 4,000, above about 4,500 or above about 5,000 psi). In exemplary embodiments, the stainless steel of the present invention possesses a tensile strength of at least about 90 ksi, at least about 100 ksi, at least about 110 ksi, or at least about 120 ksi. (or from about 90 ksi to about 200 ksi, from about 90 ksi to about 160 ksi, or from about 90 ksi to about 150 ksi). In a further exemplary embodiment, the at least one portable vessel and at least one pressure regulator are capable of a service pressure of at least about 5,000 psi and a burst pressure of at least about 10,000 psi. In an even further exemplary embodiment, the portable vessel is capable of holding at least about 80 ft³ of a gas, has a length of about 25 inches or less and an outer diameter of about 8 inches or less. Another exemplary embodiment of the present invention relates to a portable vessel that is capable of holding at least about 20 ft³ of a gas, has a length of about 10 inches or less and an outer diameter of about 5 inches or less.

In another exemplary embodiment, the present invention also relates to a stainless steel composition that provides additional improved physical properties. For example, in one embodiment of the present invention, the stainless steel possesses a high impact resistance, which provides the vessel and pressure regulator with lower brittleness at high and low temperatures (e.g., from −100 to 100° C.). This provides a stainless steel that is very durable with low susceptibility to fatigue over time, which results in a portable vessel and pressure regulator that has a long service life. In exemplary embodiments, the present invention is directed to at least one portable vessel and pressure regulator, and apparatus including the same, for storing fluids under pressure, which comprise(s) stainless steel, wherein the stainless steel possesses an impact resistance of at least about 100 J at room temperature, at least about 110 J at room temperature, at least about 120 J at room temperature, at least about 140 J at room temperature, or at least about 150 J at room temperature (or from about 100 J to about 200 J, from about 110 to about 190 J or from about 120 J to about 180 J).

In a further exemplary embodiment, the present invention is also directed to a method of storing fluid under high pressure in portable equipment. A further exemplary embodiment of the present invention relates to releasing fluid under high pressure in a portable apparatus comprising at least one portable vessel and pressure regulator, wherein the pressure regulator reduces the high pressure in the portable vessel to a lower predetermined pressure. In another exemplary embodiment, the present invention relates to a method of storing fluid in a portable vessel, including (a) filling the fluid into the portable vessel composed of stainless steel and being cylindrical in shape with closed ends, with at least one end having an aperture for receiving a valve boss; and (b) closing the valve after the fluid in the vessel reaches a pressure of at least about 4,000 psi, at least about 4,500 psi, at least about 5,000 psi, at least about 5,500 psi, or at least about 6,000 psi; or pressure ranges of from about 4,000 psi to about 10,000 psi, or from about 4,500 to about 10,000 psi, or from about 5,000 to about 10,000 psi, or from about 5,500 to about 10,000 psi, or from about 6,000 to about 10,000 psi. A further exemplary embodiment of the present invention relates to releasing fluid under a pressure of at least about 4000 psi, at least about 4,500 psi, at least about 5,000 psi, at least about 5,500 psi, or at least about 6,000 psi (or from 4,000 psi to about 10,000 psi, or from about 4,500 to about 10,000 psi, or from about 5,000 to about 10,000 psi, or from about 5,500 to about 10,000 psi, or from about 6,000 to about 10,000 psi) from a portable apparatus comprising at least one of a portable vessel and pressure regulator made from stainless steel, wherein the at least one pressure regulator reduces the pressure of at least about 4000 psi, at least about 4,500 psi, at least about 5,000 psi, at least about 5,500 psi, or at least about 6,000 psi (or 4,000 psi to about 10,000 psi, or from about 4,500 psi to about 10,000 psi, or from about 5,000 psi to about 10,000 psi, or from about 5,500 psi to about 10,000 psi, or from about 6,000 psi to about 10,000 psi) from the portable vessel to a lower predetermined pressure of about 180 psi or less, or from about 125 to about 180 psi.

In all of the above exemplary method embodiments relating to the apparatus, the portable vessel alone may be made of the stainless steel, or both the portable vessel and the pressure regulator may be made of the stainless steel. Moreover, in these embodiments, the stainless steel may be composed of at least one of the compositions recited herein, and may possess at least one of the properties recited herein. Furthermore, in these exemplary embodiments, the apparatus may include one or more portable vessels and one or more pressure regulators.

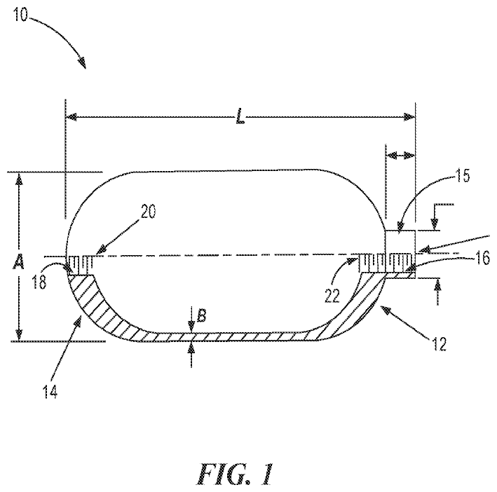

For purposes of explanation and illustration, and not limitation, a view of an exemplary embodiment of a portable vessel made in accordance with the present invention is depicted in FIG. 1. As will be described in more detail, the portable vessel of the present invention is designed to be compact compared to conventional portable vessels, while increasing capacity, corrosion resistance and fatigue resistance, since it is produced from stainless with certain chemical and physical properties, as described herein. In an exemplary embodiment, the portable vessel 10 set forth in FIG. 1 is cylindrical in nature, has a length “L”, a diameter “A” and a wall thickness “B”, which define the volume of the portable vessel 10. Even though the portable vessel 10 of this embodiment of the present invention is cylindrical in shape, other shapes may be used, such as spherical, or spheroidal, and the like. End portions, 12 and 14, are depicted as semi-hemispherical, but may be of any size or shape, depending upon the process used to form the end portions, such as, for example, semi-spheroidal, frusto-conical, flattened, rounded, or the like, and may be identical or different. In end portions 12 and 14, apertures 16 and 18 are formed, and plug boss 20 and valve boss 22 are tapped into apertures 16 and 18, respectively. A plug and K valve (not shown) are then turned into plug boss 20 and valve boss 22, respectively. In another exemplary embodiment, the end portion 14 is sealed without the use of an aperture 18 and plug boss 20.

In exemplary embodiments according to the invention, portable vessel 10 may range in size depending upon the desired capacity and intended use. For example, where a low capacity of fluid is desired, such as for an auxiliary vessel, emergency vesselor for a short-term vessel for a breathing apparatus, the portable vessel 10 capacity may range from about 5 ft³ to about 30 ft³, or from about 10 ft³ to about 25 ft³. Such vessel 10 dimensions range from about 5.0 inches to about 15.0 inches in length, and about 3.0 inches to about 6.0 inches in diameter. In exemplary embodiments where larger capacity of fluid is desired, such as for a primary portable vessel in a breathing apparatus, the portable vessel 10 capacity may range from about 30.0 ft³ to about 90.0 ft³, from about 40.0 ft³ to about 90.0 ft³ or from about 30.0 ft³ to about 90.0 ft³. Such vessel 10 dimensions range from about 15.0 inches to about 25.0 inches in length L and about 4.0 inches to about 8.0 inches in diameter A. The wall thickness B may range from about 0.05 inch to about 0.45 inch, or from about 0.08 inch to about 0.40 inch. For example, in exemplary embodiments according to the invention, the portable vessel 10 is capable of holding at least 80 ft³ of a gas, has a length L of 25.0 inches or less, and an outer diameter A of 6.0 inches or less. In exemplary embodiments according to the invention, the portable vessel 10 is capable of holding at least 20.0 ft³ of a gas, has a length L of 10.0 inches or less, and an outer diameter A of 5.0 inches or less.

In exemplary embodiments according to the invention, not only the size, but the weight of the portable vessel 10 is much lower than that of conventional portable steel vessels. For example, where a low capacity of fluid is desired, such as for an auxiliary vessel, emergency vessel, or for a short-term vessel for a breathing apparatus, the portable vessel 10 capacity may range from about 5.0 ft³ to about 30.0 ft³, or from about 10.0 ft³ to about 25.0 ft³, range from about 5.0 inches to about 15.0 inches in length L and about 3.0 inches to about 6.0 inches in diameter A, and weigh 17.0 pounds or less. In exemplary embodiments where larger capacity of fluid is desired, such as for a primary portable vessel in a breathing apparatus, the portable vessel 10 capacity may range from about 30.0 ft³ to about 90.0 ft³, from about 40.0 ft³ to about 90.0 ft³ or from about 50.0 ft³ to about 90.0 ft³, range from about 15.0 inches to about 25.0 inches in length L and about 4.0 inches to about 80 inches in diameter A, and weigh 40.0 pounds or less.

In one exemplary embodiment, the portable vessel 10 is formed by selecting a straight tube or elongate pipe having the appropriate length L, diameter A and wall thickness B that will accommodate the desired capacity of fluid at the desired service pressure. The steel elongate pipe is formed according to the process set forth in EP 3 280 826 B1, the entire subject of which is incorporated herein by reference. The elongate pipe ends are then spin formed or necked in using a hot spin forming machine such as the OSC-840, available from MJC Engineering and Technology,Inc., which bends in the pipe ends, and forms end portions 12 and 14. A neck portion 15 is formed on end portion 12. Subsequently, apertures 16 and 18 are tapped and valve boss 22 and plug boss 20 are formed in the apertures 16 and 18, respectively. In an alternative exemplary embodiment according to the invention, end portion 12 is formed without aperture 16, and the need for plug boss 20 and plug (not shown) are eliminated. In this embodiment, only aperture 16 is formed in neck portion 15 and valve boss 22 tapped into aperture 16. In a further embodiment of the present invention, the portable vessel 10 is then annealed at a temperature of at least 1500° F. A plug (not shown) is then inserted into plug boss 20 and a K valve (not shown) inserted into valve boss 22. The portable vessel 10 is then pressure tested using the hydrostatic testing method, which pressurizes the portable vessel 10 up to 3/2 or 5/3 of the working pressure, and the volume in the portable vessel 10 is measured before and after the test to determine if there is any increase in volume of the vessel. Once the pressure vessel 10 passes the hydrostatic test, it is stamped with the appropriate information (e.g., service pressure, serial number, DOT and CE specification, manufacturer, etc.) and is ready for use.

In another exemplary embodiment, the portable vessel 10 of the present invention may alternatively be manufactured from stainless steel plate discs, which are cold drawn to a cylindrical cup form, in two or three stages, and generally have a domed base. After forming the base and side walls, the top of the portable vessel 10 is trimmed to length, heated and spin formed using a spinning and necking machine, or hydro-formed, to form the shoulder and the neck. This process thickens the material of the shoulder. The vessel 10 is then machined or tapped to provide the valve boss 22.

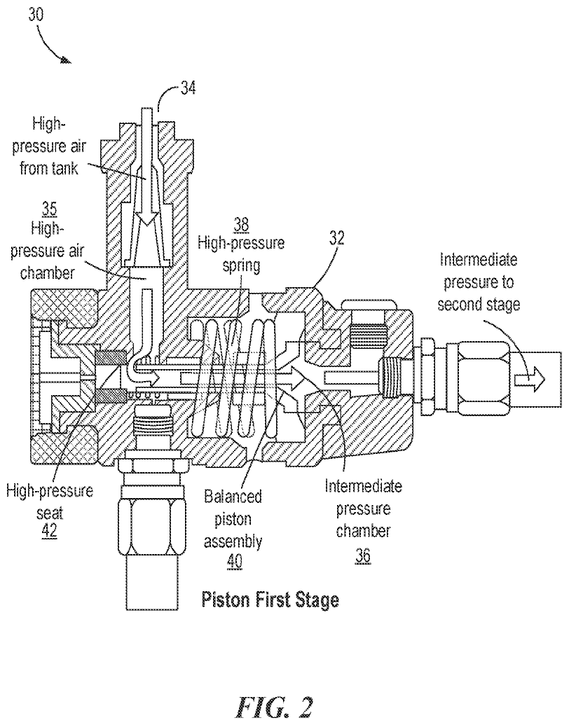

For purposes of explanation and illustration, and not limitation, a view of an exemplary embodiment of a first stage pressure regulator valve 30, made in accordance with the present invention, is depicted in FIG. 2. The first stage pressure regulator valve 30 includes housing or envelope 32, which holds all of the valve 30 components. High pressure inlet 34 and high-pressure chamber 35 are in fluid communication with a portable vessel 10 (not shown), which, during operation, receives high pressure (e.g., above about 4,000 psig or from about 4,000 to about 10,000 psig) fluid or gas from the portable vessel 10 (not shown). Once an operator inhales, the pressure in the intermediate pressure chamber 36 drops, which causes the spring 38 to expand and retract the piston 40. This results in the piston assembly 40 withdrawing from the seat 42, allowing the high-pressure fluid or gas from the high-pressure chamber 35 to flow into the intermediate pressure chamber 36, which contracts the spring 38 and forces piston assembly 40 onto the seat 42 so as to stop the flow of high-pressure fluid or gas. In one embodiment of the present invention, the housing or envelope 32 is comprised of the stainless set forth herein. The chemical and physical properties of this stainless steel allow for use of this high pressure without failure. Other components of the first stage pressure regulator valve may be formed from the stainless steel disclosed herein. Even though FIG. 2 depicts a piston first stage pressure regulator valve 30, other exemplary embodiments of the present invention include the use of different first stage pressure regulator valves, such as diaphragm first stage pressure regulator valves. In addition, the first stage pressure regulator valve 30 may be balanced or unbalanced, as is well known in the art.

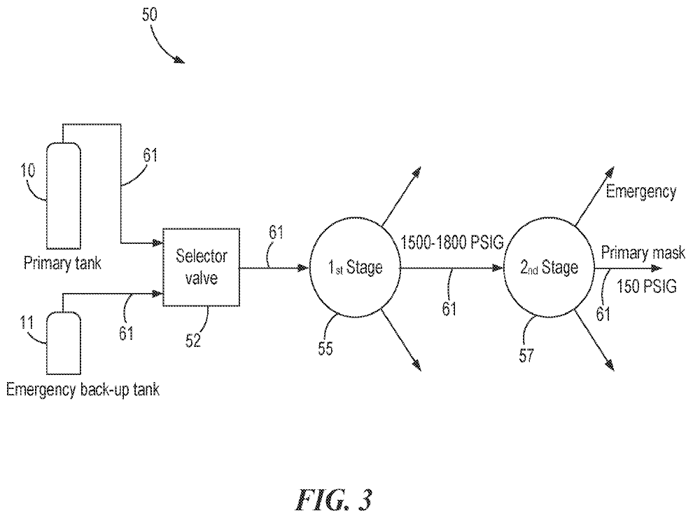

For purposes of explanation and illustration, and not limitation, a view of an exemplary embodiment of a SCUBA or SCBA apparatus, made in accordance with the present invention, is depicted as a schematic in FIG. 3. The apparatus 50 includes portable vessels 10 and 11, which may be mounted to a harness or frame (not shown) to enable the portable vessels 10 and 11 to carried on the back or side, or even the front, of a SCUBA diver or SCBA emergency personnel. Even though there are two portable vessels 10 and 11 shown in this exemplary embodiment, one or more additional portable vessels may be utilized. The portable vessels 10 and 11 may be connected to a selector valve 52, which allows the operator to switch between portable vessels 10 and 11. The selector valve 52 is connected to a first stage pressure regulator valve 55, which reduces the high pressure (e.g., above 4000 psi or from about 4,000 psi to about 10,000 psi) from the portable vessels 10 or 11 to an intermediate pressure (e.g., 1500-1800 psi). The first stage pressure regulator valve 55 is connected to a second stage pressure regulator valve 57, which reduces the pressure even further to a suitable breathing pressure (e.g., 125-180 psi). The second stage pressure regulator valve 57 may be connected to a breathing apparatus (not shown), such as a mask with a mouthpiece. The portable vessels 10 and 11, the selector valve 52, the first stage pressure regulator valve 55, the second stage pressure regulator valve 57, and the breathing apparatus (not shown) are in fluid communication with each other via one or more hoses 61. The hoses 61 and first stage pressure regulator valve 55 (or if combined with the second stage regulator valve 57) are capable of service pressures greater than about 4,000 psi, about 4,500 psi, or about 5,000 psi (or from about 4,000 to about 9,000 psi, about 4,500 to about 9,500 psi or from about 5,000 to about 10,000 psi). In the exemplary embodiment where a single portable vessel 10 is used in the apparatus 50, the portable vessel 10 is attached directly to the first stage pressure regulator valve 55 via hose 61, without the need for a selector valve 52. In such a configuration, the first stage pressure regulator valve 55 may, alternatively, be attached directly to the portable vessel 10, without the need for hose 61. In another exemplary embodiment, the first stage pressure regulator valve 55 may be combined with the second stage pressure regulator valve 57 in a single housing (not shown).

In other exemplary embodiments according to the present invention, the second stage pressure regulator valve 57, described above with regard to FIG. 3, may include diaphragm regulator valves, such as Mikron or Titan available from Aqua Lung International; or piston regulator valves, such as Scubapro S560 or A700 available from Johnson Outdoors. Such valves 57 may be balanced or unbalanced, include downstream or pilot valves, be adjustable or un-adjustable, and include venture assist, as is well known in the art. Additionally, the second stage pressure regulator may be part of the breathing device (e.g., mask), or may separate but in fluid communication with the breathing device.

In other exemplary embodiments according to the present invention, the apparatus 50 set forth in FIG. 3 may also include a dive computer (not shown) that measures the pressure in the one of more portable vessels (10 and 11) such that the operator may determine how much time or gas is left in the portable vessel(s) (10 and 11) without the need to look at a pressure gauge on the portable vessel(s) (10 and 11). The computer is wirelessly connected to the portable vessel(s) via a transmitter attached to the portable vessel(s) (10 and 11), which transmits the portable vessel(s) pressure to the computer. In exemplary embodiments according to the invention, the computer, when used for SCUBA applications, measures depths, decompression status and maximum depth, bottom time, descent and ascent rates, ambient temperature, surface intervals, and all warning points entered during the dive. Such a dive computer includes the Eon Core or DX available from Suunto Oy; the i7770R available from Aqua Lung International; or the Scubapro Galileo HUD available from Johnson Outdoors, Inc.

### Additional Embodiments

1. A portable vessel for storing fluids under pressure, said portable vessel comprising stainless steel, wherein said stainless steel (1) comprises ferritic and austenitic phases, and (2) comprises at least the following: chromium in an amount of about 17.0 to about 40.0 weight percent (wt %); molybdenum in an amount of about 0.01 to about 10.00 wt %; and nickel in an amount of about 1.0 to about 12.0 wt %, based on a total weight of the stainless steel.

2. A portable vessel for storing fluids under pressure, said portable vessel comprising stainless steel, wherein said stainless steel (1) comprises ferritic and austenitic phases, and (2) possesses a tensile strength of at least 90 ksi.

3. A portable vessel for storing fluids under pressure, said portable vessel comprising stainless steel, wherein said stainless steel (1) comprises ferritic and austenitic phases, and (2) possesses a critical pitting corrosion temperature according to ASTM G48C of at least 80° C.

4. A portable vessel for storing fluids under pressure, said portable vessel comprising stainless steel, wherein said stainless steel (1) comprises ferritic and austenitic phases, and (2) possesses a pitting resistance equivalent number of at least 30, wherein the pitting resistance equivalent (PRE) number is calculated using the following formula:

PRE=(wt % Cr)+3.3×(wt % Mo)+16×(wt % N)

wherein wt % is weight percent, Cr is chromium, Mo is molybdenum and N is nitrogen.

5. A portable vessel for storing fluids under pressure, said portable vessel comprising stainless steel, wherein said stainless steel (1) comprises ferritic and austenitic phases, and (2) possesses an impact resistance of at least 100 J at room temperature.

6. A portable vessel for storing fluids under pressure, said portable vessel comprising stainless steel, wherein said stainless steel (1) comprises ferritic and austenitic phases, and (2) comprises at least the following: chromium in an amount of about 17.0 to about 40.0 weight percent (wt %); molybdenum in an amount of about 0.01 to about 10.00 wt %; and nickel in an amount of about 1.0 to about 12.0 wt %, based on a total weight of the stainless steel, and wherein said portable vessel is capable of a service pressure of at least 4,000 psi and a burst pressure of at least 9,000 psi.

7. A portable vessel according to any one of embodiments 2 to 5, wherein said stainless steel comprises: chromium in an amount of about 17.0 to about 40.0 wt %; molybdenum in an amount of about 0.01 to about 10.00 wt %; and nickel in an amount of about 1.0 to about 12.0 wt %, based on a total weight of the stainless steel.

8. A portable vessel according to any one of embodiments 1 to 7, wherein said stainless steel comprises: chromium in an amount of about 20.0 to about 40.0 wt %; molybdenum in an amount of about 0.05 to about 10.00 wt %; and nickel in an amount of about 1.5 to about 12.0 wt %, based on a total weight of the stainless steel.

9. A portable vessel according to any one of embodiments 1 to 8, wherein said stainless steel comprises: chromium in an amount of at least about 25.0 wt %; molybdenum in an amount of at least about 3.6 wt %; and nickel in an amount of at least about 6.0 wt %, based on a total weight of the stainless steel.

10. A portable vessel according to any one of embodiments 1 to 9, wherein said stainless steel comprises: copper in an amount of greater than 0 up to about 1.0 wt %; nitrogen in an amount of at least about 0.25 wt %; and silicon in amount of greater than 0 up to about 0.6 wt %.

11. A portable vessel according to any one of embodiments 1 to 10, wherein said stainless steel comprises at least the following: chromium in an amount of from about 25.0 to about 40.0 wt %; molybdenum in an amount of from about 3.6 to 10.00 wt %; nickel in an amount of about 6.0 to about 12.0 wt %; copper in an amount of greater than 0 up to about 0.25 wt %; nitrogen in an amount of from about 0.25 to about 0.6 wt %; and silicon in amount of from about 0.1 to about 0.4 wt %, based on a total weight of the stainless steel.

12. A portable vessel according to any one of embodiments 1 to 11, wherein said stainless steel comprises at least the following: chromium in an amount of about 25.38 wt %; molybdenum in an amount of about 3.88 wt %; nickel in an amount of about 6.44 wt %; copper in an amount of about 0.12 wt %; nitrogen in an amount of about 0.299 wt %; and silicon in amount of about 0.28 wt %.

13. A portable vessel according to any one of embodiments 1 to 12, wherein said stainless steel further comprises: carbon in an amount of greater than 0 up to about 0.25 wt %; iron in an amount of from about 50.0 to about 75.0 wt %; manganese in an amount of greater than 0 up to about 1.0 wt %; phosphorous in an amount of greater than 0 up to about 0.05 wt %; and sulfur in an amount of greater than 0 up to about 0.03 wt %, based on a total weight of the stainless steel.

14. A portable vessel according to any one of embodiments 1 to 13, wherein said stainless steel further comprises: carbon in an amount of greater than 0 up to about 0.01 wt %; iron in an amount of from about 55.0 to about 73.0 wt %; manganese in an amount of greater than 0 up to about 0.7 wt %; phosphorous in an amount of greater than 0 up to about 0.03 wt %; and sulfur in an amount of greater than 0 up to about 0.01 wt %, based on a total weight of the stainless steel.

15. A portable vessel according to any one of embodiments 1 to 14, wherein said stainless steel further comprises: carbon in an amount of about 0.014 wt %; iron in an amount of from about 55.2 to about 72.7 wt %; manganese in an amount of about 0.49 wt %; phosphorous in an amount of about 0.019 wt %; and sulfur in an amount of about 0.0009 wt %.

16. A portable vessel according to any one of embodiments 1 and 3 to 15, wherein said stainless steel possesses a tensile strength of at least 90 ksi.

17. A portable vessel according to any one of embodiments 1 to 16, wherein said stainless steel possesses a tensile strength of at least 110 ksi.

18. A portable vessel according to any one of embodiments 1 to 2 and 4 to 17, wherein said stainless steel possesses a critical pitting corrosion temperature according to ASTM G48C of at least 80° C.

19. A portable vessel according to any one of embodiments 1 to 18, wherein said stainless steel possesses a critical pitting corrosion temperature according to ASTM G48C of at least 85° C.

20. A portable vessel according to any one of embodiments 1 to 19, wherein said stainless steel possesses a pitting resistance equivalent number of at least 33.

21. A portable vessel according to any one of embodiments 1 to 20, wherein said stainless steel possesses a pitting resistance equivalent number of at least 35.

22. A portable vessel according to any one of embodiments 1 to 21, wherein said stainless steel possesses a pitting resistance equivalent number of at least 38.

23. A portable vessel according to any one of embodiments 1 to 22, wherein said stainless steel possesses a pitting resistance equivalent number of at least 40.

24. A portable vessel according to any one of embodiments 1 to 23, wherein said stainless steel possesses a pitting resistance equivalent number of at least 41.5.

25. A portable vessel according to any one of embodiments 1 to 24, wherein said stainless steel possesses a pitting resistance equivalent number of about 41.8.

26. A portable vessel according to any one of embodiments 1 to 4 and 6 to 25, wherein said stainless steel possesses an impact resistance of at least 100 J at room temperature.

27. A portable vessel according to any one of embodiments 1 to 26, wherein said stainless steel possesses an impact resistance of at least 120 J at room temperature.

28. A portable vessel according to any one of claims 1 to 27, wherein said stainless steel has a ferritic phase represents from about 30 to about 70 volume percent, and the austenitic phase represents from about 70 to about 30 volume percent.

29. A portable vessel according to any one of claims 1 to 28, wherein said stainless steel has a ferritic phase represents from about 40 to about 60 volume percent, and the austenitic phase represents from about 60 to about 40 volume percent.

30. A portable vessel according to any one of embodiments 1 to 5 and 7 to 29, wherein said portable vessel is capable of a service pressure of at least 4,000 psi and a burst pressure of at least 9,000 psi.

31. A portable vessel according to any one of embodiments 1 to 30, wherein said portable vessel is capable of a service pressure of at least 4,500 psi and a burst pressure of at least 9,500 psi.

32. A portable vessel according to any one of embodiments 1 to 31, wherein said vessel is cylindrical in shape.

33. A portable vessel according to any one of embodiments 1 to 32, wherein said vessel is cylindrical in shape with closed ends.

34. A portable vessel according to any one of embodiments 1 to 33, wherein said vessel is cylindrical in shape with closed ends, and at least one end has an aperture for receiving a valve boss.

35. A portable vessel according to any one of embodiments 1 to 34, wherein said vessel is cylindrical in shape with closed ends, and at least one end has an aperture for receiving a valve boss, and an opposite end has an aperture therein for receiving a plug boss.

36. A portable vessel according to any one of embodiments 1 to 35, wherein said vessel has a length of about 25.0 inches or less, and an outer diameter of about 8.0 inches or less.

37. A portable vessel according to any one of embodiments 1 to 36, wherein said vessel has a length of from about 5.0 to about 25.0 inches, and an outer diameter of from about 3.0 to about 6.0 inches.

38. A portable vessel according to any one of embodiments 1 to 37, wherein said vessel has a length of 10.0 inches or less, and an outer diameter of 5.0 inches or less.

39. A portable vessel according to any one of embodiments 1 to 38, wherein said vessel has a wall thickness of from about 0.05 inch to about 0.45 inch.

40. A portable vessel according to any one of embodiments 1 to 39, wherein said vessel has a wall thickness of from about 0.08 inch to about 0.40 inch.

41. A portable vessel according to any one of embodiments 1 to 40, wherein said vessel is capable of holding at least about 20.0 ft³ of a gas (e.g., a breathable gas).

42. A portable vessel according to any one of embodiments 1 to 37 and 39 to 41, wherein said vessel is capable of holding at least about 80.0 ft³ of a gas.

43. A portable vessel according to any one of embodiments 1 to 37 and 39 to 42, wherein said vessel is capable of holding up to about 90.0 ft³ of a gas (e.g., a breathable gas).

44. A portable vessel according to any one of embodiments 1 to 41, wherein said vessel is cylindrical in shape with closed ends, and at least one end having an aperture for receiving a valve boss; and wherein said vessel is capable of holding at least 20.0 ft³ of a gas, has a length of 10.0 inches or less, and an outer diameter of 5.0 inches or less.

45. A portable vessel according to any one of embodiments 1 to 37 and 39 to 43, wherein said vessel is cylindrical in shape with closed ends, and at least one end having an aperture for receiving a valve boss; and wherein said vessel is capable of holding at least 80.0 ft³ of a gas, has a length of 25.0 inches or less, and an outer diameter of 6.0 inches or less.

46. A portable vessel according to any one of embodiments 1 to 45, wherein said vessel weighs 40.0 pounds or less.

47. A portable vessel according to any one of embodiments 1 to 41 and 44 to 46, wherein said vessel weighs 17.0 pounds or less.

48. A portable vessel according to any one of embodiments 1 to 47, wherein said vessel comprises one or more tank walls extending from one end to an opposite end, and each of said one or more tank walls comprises a continuous, single layer of said stainless steel.

49. A portable vessel according to any one of embodiments 1 to 48, wherein said vessel comprises a single tank wall extending from one end to an opposite end, and said single tank wall comprises a continuous, single layer of said stainless steel.

50. An apparatus for storing fluids under pressure, said apparatus comprising: (I) at least one portable vessel according to any one of embodiments 1 to 49, wherein the at least one portable vessel is cylindrical in shape with closed ends, with at least one end having an aperture for receiving a valve boss; and (II) at least one pressure regulator, which reduces the pressure from said at least one portable vessel to a predetermined level.

51. An apparatus according to embodiment 50, wherein the at least one pressure regulator comprises the stainless steel recited in any one of embodiments 1 to 29.

52. An apparatus according to embodiment 50 or 51, wherein said at least one portable vessel comprises (i) at least one first portable vessel that is cylindrical in shape with closed ends, with at least one end having an aperture for receiving a valve boss, and (ii) at least one second portable vessel that is cylindrical in shape with closed ends, with at least one end having an aperture for receiving a valve boss, said at least one second portable vessel being different in size from said at least one first portable vessel.

53. An apparatus according to embodiment 52, wherein (i) said at least one first portable vessel is capable of holding at least 80.0 ft³ of a gas, has a length of 25.0 inches or less, and an outer diameter of 8.0 inches or less, and (ii) said at least one second portable vessel is capable of holding at least 20.0 ft³ of a gas, has a length of 10.0 inches or less, and an outer diameter of 5.0 inches or less.

54. An apparatus according to embodiment 52 or 53, wherein said at least one portable vessel comprises (i) at least one first portable vessel that is cylindrical in shape with closed ends, with at least one end having an aperture for receiving a valve boss, and (ii) at least one second portable vessel that is cylindrical in shape with closed ends, with at least one end having an aperture for receiving a valve boss, said at least one second portable vessel being essentially identical in size from said at least one first portable vessel.

55. A pressure regulator for reducing pressure from at least one portable vessel capable of storing fluids under pressure, said pressure regulator comprising: a valve comprising a valve body, said valve body being comprised of stainless steel, to be in fluid communication with said at least one portable vessel, which reduces the pressure from said at least one portable vessel to a predetermined level; wherein said stainless steel comprises the stainless steel recited in any one of embodiments 1 to 29.

56. A method of storing fluid in a portable vessel, said method comprising: inputting the fluid into the portable vessel, the portable vessel comprising the portable vessel according to any one of embodiments 1 to 49.

57. The method of embodiment 56, further comprising closing a valve after the fluid in the portable vessel reaches a pressure of at least about 4,000 psi.

58. The method of embodiment 56 or 57, further comprising closing a valve after the fluid in the portable vessel reaches a pressure of at least about 4,500 psi.

59. The method of any one of embodiments 56 to 58, further comprising closing a valve after the fluid in the portable vessel reaches a pressure of at least about 5,000 psi.

60. The method of any one of embodiments 56 to 59, further comprising closing a valve after the fluid in the portable vessel reaches a pressure of at least about 5,500 psi.

61. A method of making the portable vessel of any one of embodiments 1 to 49, said method comprising: forming a melt of the stainless steel; casting the melt into one or more billets; extruding the one or more billets to form a tube; subjecting the tube to a cold deformation process where the tube is cold rolled without heat in a pilger mill; annealing the tube at about 1900° F.; and rapidly quenching and picking.

### Examples

The present invention is further illustrated by the following examples, which are not to be construed in any way as imposing limitations upon the scope thereof. On the contrary, it is to be clearly understood that resort may be had to various other exemplary embodiments, modifications, and equivalents thereof which, after reading the description herein, may suggest themselves to those skilled in the art without departing from the spirit of the present invention and/or the scope of the appended claims.

### Examples 1 and 2

In these examples, two portable vessels, in this case cylinders, are prepared from stainless steel having the following composition: carbon in an amount of 0.014 wt %; chromium in an amount of 25.38 wt %; copper in an amount of 0.12 wt %; iron in an amount of 55.2-72.7 wt %; manganese in an amount of 0.49 wt %; molybdenum in an amount of 3.88 wt %; nickel in an amount of 6.44 wt %; nitrogen in an amount of 0.299 wt %; phosphorous in an amount of 0.019 wt %; silicon in amount of 0.28 wt %; and sulfur in an amount of 0.0009 wt %, with the remainder being iron and naturally occurring impurities. Melts of stainless steels of the present invention are prepared in an electric arc furnace. An AOD (argon oxygen decarburization) furnace is used in which decarburization and desulfurization treatment are employed. The melt is cast into billets and then subjected to a heat deformation process in which the billets are extruded into tubes at a temperature of 2000° F. for two minutes. The tubes are then subjected to a cold deformation process where they are cold rolled (i.e., without heating) in a pilger mill into their final dimensions. The tubes are then annealed in a furnace at 1900° F. followed by rapid quenching and pickling. The stainless steel has a tensile strength of 94.54 ksi and a tensile strength of 122.8 ksi. The tubes are then cut to the desired length and formed into cylindrical vessels by manually closing the ends using a roller on a metal spin forming machine or lathe. The vessels are 16 inches in length, 4.5 inches in diameter, and have a wall thickness of 0.25 inch. The vessels have a capacity of 160 in³. The vessels are then pressure tested according to the DOT (Department of Transportation) approved hydrostatic pressure test. The vessels have a proof pressure of 10,533 psi and a burst pressure of 12,450 psi. The vessels have a service pressure of 4,500 psi and are filled to 55.5 ft³ of air and weigh 22.2 lbs. These examples demonstrate that the portable vessels of the present invention are very compact, hold substantial volumes of fluid, and operate at very high pressures.

While the invention has been described with a limited number of exemplary embodiments, these specific exemplary embodiments are not intended to limit the scope of the invention as otherwise described and claimed herein. It may be evident to those of ordinary skill in the art upon review of the exemplary embodiments herein that further modifications, equivalents, and variations are possible. All parts and percentages in the examples, as well as in the remainder of the specification, are by weight unless otherwise specified. Further, any range of numbers recited in the specification or claims, such as that representing a particular set of properties, units of measure, conditions, physical states or percentages, is intended to literally incorporate expressly herein by reference or otherwise, any number falling within such range, including any subset of numbers within any range so recited. For example, whenever a numerical range with a lower limit, R_{L}, and an upper limit R_{U}, is disclosed, any number R falling within the range is specifically disclosed. In particular, the following numbers R within the range are specifically disclosed: R=R_{L}+k(R_{U}−R_{L}), where k is a variable ranging from 1% to 100% with a 1% increment, e.g., k is 1%, 2%, 3%, 4%, 5% . . . 50%, 51%, 52% . . . 95%, 96%, 97%, 98%, 99%, or 100%. Moreover, any numerical range represented by any two values of R, as calculated above is also specifically disclosed. Any modifications of the invention, in addition to those shown and described herein, will become apparent to those skilled in the art from the foregoing description and accompanying drawings. Such modifications are intended to fall within the scope of the appended claims. All publications cited herein are incorporated by reference in their entirety.

1. A portable self-contained breathing apparatus vessel for storing breathable gas under pressure, said portable vessel comprising stainless steel, wherein said stainless steel (1) comprises ferritic and austenitic phases, (2) comprises a tensile strength of at least 90 ksi, and (3) comprises a pitting resistance equivalent number of at least 30, wherein the pitting resistance equivalent (PRE) number is calculated using the following formula:
PRE=(wt % Cr)+3.3×(wt % Mo)+16×(wt % N)
wherein wt % is weight percent, Cr is chromium, Mo is molybdenum and N is nitrogen.

2. A portable self-contained breathing apparatus vessel according to claim 1, wherein said stainless steel comprises: chromium in an amount of about 17.0 to about 40.0 wt %; molybdenum in an amount of about 0.01 to about 10.00 wt %; and nickel in an amount of about 1.0 to about 12.0 wt %, based on a total weight of the stainless steel.

3-11. (canceled)

12. A portable self-contained breathing apparatus vessel according to claim 1, wherein said stainless steel possesses a tensile strength of at least 100 ksi.

13. A portable self-contained breathing apparatus vessel according to claim 1, wherein said stainless steel possesses a critical pitting corrosion temperature according to ASTM G48C of at least 80° C.

14. (canceled)

15. A portable self-contained breathing apparatus vessel according to claim 1, wherein said stainless steel possesses a pitting resistance equivalent number of at least 33.

16-20. (canceled)

21. A portable self-contained breathing apparatus vessel according to claim 1, wherein said stainless steel possesses an impact resistance of at least 100 J at room temperature.

22. (canceled)

23. A portable self-contained breathing apparatus vessel according to claim 1, wherein said stainless steel has a ferritic phase represents from about 30 to about 70 volume percent, and the austenitic phase represents from about 70 to about 30 volume percent.

24. (canceled)

25. A portable self-contained breathing apparatus vessel according to claim 1, wherein said portable self-contained breathing apparatus vessel is capable of a service pressure of at least 4,000 psi and a burst pressure of at least 9,000 psi.

26-30. (canceled)

31. A portable self-contained breathing apparatus vessel according to claim 1, wherein said portable self-contained breathing apparatus vessel has a length of about 25.0 inches or less, and an outer diameter of about 8.0 inches or less is capable of holding at least about 80.0 ft³ of a gas.

32. A portable self-contained breathing apparatus vessel according to claim 1, wherein said portable self-contained breathing apparatus vessel has a length of from about 5.0 to about 25.0 inches, and an outer diameter of from about 3.0 to about 8.0 inches.

33. A portable self-contained breathing apparatus vessel according to claim 1, wherein said portable self-contained breathing apparatus vessel has a length of 10.0 inches or less, and an outer diameter of 5.0 inches or less and is capable of holding at least about 20.0 ft³ of a gas.

34. A portable self-contained breathing apparatus vessel according to claim 1, wherein said portable self-contained breathing apparatus vessel has a wall thickness of from about 0.05 inch to about 0.45 inch.

35. (canceled)

36. A portable self-contained breathing apparatus vessel according to claim 1, wherein said portable self-contained breathing apparatus vessel weighs 40.0 pounds or less.

37. A portable self-contained breathing apparatus vessel according to claim 1, wherein said portable self-contained breathing apparatus vessel weighs 17.0 pounds or less.

38-39. (canceled)

40. An apparatus for storing breathable gas under pressure, said apparatus comprising:
at least one portable self-contained breathing apparatus vessel according to claim 1, wherein the at least one portable self-contained breathing apparatus vessel is cylindrical in shape with closed ends, with at least one end having an aperture for receiving a valve boss; and
at least one pressure regulator, which reduces the pressure from said at least one portable self-contained breathing apparatus vessel to a predetermined level.

41. An apparatus according to claim 40, wherein the at least one pressure regulator comprises stainless steel that (1) comprises ferritic and austenitic phases, (2) comprises a tensile strength of at least 90 ksi, and (3) comprises a pitting resistance equivalent number of at least 30, wherein the pitting resistance equivalent (PRE) number is calculated using the following formula:
PRE=(wt % Cr)+3.3×(wt % Mo)+16×(wt % N)
wherein wt % is weight percent, Cr is chromium, Mo is molybdenum and N is nitrogen.

42. An apparatus according to claim 40, wherein said at least one portable self-contained breathing apparatus vessel comprises (i) at least one first portable self-contained breathing apparatus vessel that is cylindrical in shape with closed ends, with at least one end having an aperture for receiving a valve boss, and (ii) at least one second portable self-contained breathing apparatus vessel that is cylindrical in shape with closed ends, with at least one end having an aperture for receiving a valve boss, said at least one second portable self-contained breathing apparatus vessel being different in size from said at least one first portable self-contained breathing apparatus vessel.

43. An apparatus according to claim 42, wherein (i) said at least one first portable self-contained breathing apparatus vessel is capable of holding at least 80.0 ft³ of a gas, has a length of 25.0 inches or less, and an outer diameter of 8.0 inches or less, and (ii) said at least one second portable self-contained breathing apparatus vessel is capable of holding at least 20.0 ft³ of a gas, has a length of 10.0 inches or less, and an outer diameter of 5.0 inches or less.

44. An apparatus according to claim 40, wherein said at least one portable self-contained breathing apparatus vessel comprises (i) at least one first portable self-contained breathing apparatus vessel that is cylindrical in shape with closed ends, with at least one end having an aperture for receiving a valve boss, and (ii) at least one second portable self-contained breathing apparatus vessel that is cylindrical in shape with closed ends, with at least one end having an aperture for receiving a valve boss, said at least one second portable self-contained breathing apparatus vessel being essentially identical in size from said at least one first portable self-contained breathing apparatus vessel.

45. A pressure regulator for reducing pressure from at least one portable self-contained breathing apparatus vessel capable of storing breathable gas under pressure, said pressure regulator comprising:
a valve comprising a valve body, said valve body being comprised of stainless steel, to be in fluid communication with said at least one portable self-contained breathing apparatus vessel, which reduces the pressure from said at least one portable self-contained breathing apparatus vessel to a predetermined level;
wherein said stainless steel comprises the stainless steel recited in claim 1.
